(19) United States
(12) Patent Application Publication
(10) Pub. No.: US 2008/0064760 A1
(43) Pub. Date: Mar. 13, 2008

(54) Spontaneously Dispersible Pharmaceutical Composition

(76) Inventors: Ping Li, Basking Ridge, NJ (US); Madhusudhan Pudipeddi, Edison, NJ (US); Alan Edward Royce, Saylorsburg, PA (US); Sara Royce Hynes, New Haven, CT (US); Abu T.M. Serajuddin, Queens, NY (US); Michael Ambuhl, Rheinfelden (CH); Barbara Luckel, Lorrach (DE)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/575,203

(22) PCT Filed: Oct. 27, 2005
(86) PCT No.: PCT/US05/39004

§ 371 Date: Mar. 13, 2007

Related U.S. Application Data
(60) Provisional application No. 60/623,345, filed on Oct. 29, 2004.
(60) Provisional application No. 60/623,344, filed on Oct. 29, 2004.

(51) Int. Cl. A61K 47/00

(52) U.S. Cl. 514/772.1

(57) ABSTRACT

A spontaneously dispersible pharmaceutical composition comprising a poorly soluble drug and a carrier medium comprising (1) a lipophilic component, (2) a surfactant, and optionally (3) a hydrophilic component, wherein at least one of the components (1) to (3) is solid at room temperature. A particularly useful hydrophilic component in the system is a polymer that is solid at room temperature, e.g., solid PEG.

### FIELD OF THE INVENTION

The present invention relates to a pharmaceutical composition, e.g. a microemulsion preconcentrate that includes a drug in a solid or semisolid carrier. The system forms an emulsion, e.g., a microemulsion when brought in contact with an aqueous medium, e.g., water or the gastric juices of the gastrointestinal tract. Especially useful in the present invention are, e.g., drugs that are poorly soluble in water.

### BACKGROUND OF THE INVENTION

A particularly useful vehicle for administering a drug to a mammal, e.g., a human, is a microemulsion preconcentrate. A microemulsion preconcentrate, e.g., includes at least one oil or other lipophilic ingredients, at least one surfactant, optional hydrophilic ingredients, and any other agents or excipients as needed. When the components of the system contact an aqueous medium, e.g., water, a microemulsion spontaneously forms, such as an oil-in-water (o/w) microemulsion, with little or no agitation. The resulting microemulsion is a thermodynamically stable system comprising two immiscible liquids, in which one liquid is finely divided into the other because of the presence of a surfactant(s). The microemulsion formed, e.g., appears clear or translucent, slightly opaque, opalescent, non-opaque or substantially non-opaque because of the low particle size of the dispersed phase.

These drug delivery systems of the present invention can be ingested with the expectation that an emulsion, e.g., a microemulsion, forms in the gastrointestinal tract. Possible benefits of such a system include, but are not limited to, the enhanced bioavailability of the drug.

Poorly water-soluble drugs may be difficult to administer and formulate because of their low dissolution rate, low bioavailability, food effect and variability in inter- and intra-patient dose response. An exemplary method of minimizing such difficulties is to formulate such drugs as microemulsion preconcentrates. Once these systems form microemulsions in the gastrointestinal fluids after oral intake, the drug usually remains solubilized in the lipid or hydrophobic phase of the microemulsion and/or in the micellar phase of the surfactant. However, one disadvantage of the microemulsion preconcentrate as a drug delivery system is that it is often administered in a concentrated liquid form either as a drink solution or by encapsulation in a soft elastic capsule. Thus, there remains a need for a microemulsion preconcentrate that can be administered in a solid or semisolid state, e.g., as tablets, powders or filled directly in gelatin capsules, e.g., hard or soft gelatin. Such a solid or semisolid system may offer better handling and processing characteristics, as well as patient convenience.

It has now been surprisingly found that particularly suitable compositions containing poorly water-soluble drugs having, for example, particularly interesting bioavailability characteristics and reduced variability in inter- and intra-subject bioavailability parameters are obtainable using a carrier having (1) a lipophilic component, (2) a surfactant, and optionally (3) a hydrophilic component, wherein at least one of the components (1) to (3) is solid at room temperature.

### SUMMARY OF THE INVENTION

A pharmaceutical composition, e.g. a microemulsion preconcentrate, is disclosed herein. The microemulsion preconcentrate contains a drug, such as a poorly water-soluble drug, within a carrier that contains a (1) lipophilic component, (2) a surfactant, and optionally (3) a hydrophilic component, wherein at least one of the components (1) to (3) is solid at room temperature.

The microemulsion preconcentrate, at room temperature is solid or semisolid. When brought into contact with an aqueous medium, e.g., gastric juices, it forms a microemulsion with the aqueous medium. For example, an o/w microemulsion is formed with the aqueous medium being the external phase. The internal phase contains at least the lipophilic component, and the drug being delivered may be present in or mixed with the internal phase or at the surface of the internal phase.

In one exemplary embodiment of the present invention, the lipophilic component is a liquid lipophilic component, e.g., an essential oil. In a particular aspect of the present invention, a hydrophilic component that is a solid polymer at room temperature is included.

Particularly useful as a solid hydrophilic polymer are solid polyoxyethylene glycols. Examples of solid polyethylene glycols (PEGS) include, but are not limited to, PEG 1450, PEG 3350, PEG 4000, PEG 8000 and combinations and mixtures thereof. In another aspect of the present invention, the emulsion formed is a microemulsion having particles that have a mean particle size of about 50 nm to about 300 nm.

Another exemplary embodiment of the present invention is a process for preparing a microemulsion containing a poorly soluble drug. Such a process, e.g., includes the steps of bringing a drug and a liquefied carrier having a surfactant, a lipophilic component and optionally a hydrophilic component into intimate admixture to form a pharmaceutical composition. The resulting composition is, e.g., solid or semisolid at room temperature. The pharmaceutical composition is then subsequently brought into contact with an aqueous medium to form a microemulsion.

### DETAILED DESCRIPTION OF THE INVENTION

The present invention relates to a pharmaceutical composition, i.e., a microemulsion preconcentrate, that includes a drug in a carrier that comprises a lipophilic component, a surfactant and an optional hydrophilic component in a solid or semisolid form. When the pharmaceutical composition is brought into contact with an aqueous medium, an emulsion, especially a microemulsion, spontaneously forms. In particular, a microemulsion forms in the digestive tract of a mammal when the delivery system of the present invention is orally ingested. In addition to the aforementioned components, the microemulsion preconcentrate can also optionally contain other excipients, such as buffers, pH adjusters, stabilizers and other adjuvants recognized by one of ordinary skill in the art to be appropriate for such a pharmaceutical use.

A pharmaceutical composition is “pharmaceutically acceptable” which refers to those compounds, materials, compositions and/or dosage forms, which are, within the scope of sound medical judgment, suitable for contact with the tissues of mammals, especially humans, without excessive toxicity, irritation, allergic response and other problem complications commensurate with a reasonable benefit/risk.

As used herein, the term “drug” means any compound, substance, drug, medicament or active ingredient having a therapeutic or pharmacological effect, and which is suitable for administration to a mammal, e.g., a human. Such drugs should be administered in a “therapeutically effective amount”.

As used herein, the term “therapeutically effective amount” refers to an amount or concentration which is effective in reducing, eliminating, treating, preventing or controlling the symptoms of a disease or condition affecting a mammal. The term “controlling” is intended to refer to all processes wherein there may be a slowing, interrupting, arresting or stopping of the progression of the diseases and conditions affecting the mammal. However, “controlling” does not necessarily indicate a total elimination of all disease and condition symptoms, and is intended to include prophylactic treatment.

The appropriate therapeutically effective amount is known to one of ordinary skill in the art as the amount varies with the therapeutic compound being used and the indication which is being addressed.

Drugs that are particularly suited for the present invention are those that are poorly soluble or insoluble in water. As used herein, the term “poorly water-soluble” or “poorly soluble” refers to having a solubility in water at 20° C. of less than 1%, i.e., a “sparingly soluble to practically insoluble, or insoluble drug” as described in Remington, The Science and Practice of Pharmacy, 21^{st} Edition, p. 212 D. B. Troy, Ed., Lippincott Williams & Wilkins (2005).

The drug may be present in an amount up to about 20% by weight of the composition, from about 0.5% to about 15% by weight of the composition, or from about 1.5% to 10% by weight of the composition. It is intended, however, that the choice of a particular level of drug will be made in accordance with factors well-known in the pharmaceutical arts, including the solubility of the drug in the lipophilic component or optional hydrophilic component or surfactant used, mode of administration and the size and condition of the subject.

Examples of therapeutic classes of drugs include, but are not limited to, anti-hypertensives, anti-anxiety agents, anti-clotting agents, anti-convulsants, blood glucose-lowering agents, decongestants, anti-histamines, anti-tussives, anti-neoplastics, beta-blockers, anti-inflammatories, anti-psychotic agents, cognitive enhancers, anti-atherosclerotic agents, cholesterol reducing agents, anti-obesity agents, autoimmune disorder agents, anti-impotence agents, anti-bacterial and anti-fungal agents, hypnotic agents, antibiotics, anti-depressants, antiviral agents and combinations of the foregoing.

Particularly useful as poorly water-soluble drugs are cyclosporines. Cyclosporines to which the present invention can apply are any of those having pharmaceutical utility, e.g., as immunosuppressive agents, anti-parasitic agents, and agents for the reversal of multi-drug resistance. Such cyclosporines include, without limitation, Cyclosporine A (also known as Ciclosporin), Cyclosporine G, [O-(2-hydroxyethyl)-(D)Ser]8-Ciclosporin and [3′-dehydroxy-3′-keto-MeBmt]1-(Val]2-Ciclosporin. The dose of cyclosporine in the compositions of the present invention is of the same order as, or up to half of that which is used in known compositions containing cyclosporine. The optimal dosage of drug to be administered to a particular patient may be considered carefully as individual response to and metabolism of the drug, e.g. cyclosporine, may vary, e.g. by monitoring the blood serum levels of the drug by radioimmunoassay (RIA), enzyme linked immunosorbent assay (ELISA), or other appropriate conventional means. Cyclosporine doses may be 25 to 1000 mg per day (preferably 50 mg to 500 mg).

Pharmaceutical compositions comprising a cyclosporine are particularly useful for:

a) treatment and prevention of organ or tissue transplant rejection, for example for the treatment of the recipients of heart, lung, combined heart-lung, liver, kidney, pancreatic, skin or corneal transplants. The pharmaceutical compositions are also indicated for the prevention of graft-versus-host disease, such as sometimes occurs following bone marrow transplantation; and

b) treatment and prevention of autoimmune disease and of inflammatory conditions, in particular inflammatory conditions with an etiology including an autoimmune component such as arthritis (for example rheumatoid arthritis, arthritis chronic progrediente and arthritis deformans) and rheumatic diseases.

As used herein, the term “carrier” refers to the pharmaceutically acceptable vehicle that transports the drug across the biological membrane or within a biological fluid. The carrier, of the present invention, comprises a lipophilic component, optionally a hydrophilic component and a surfactant. The carrier of the present invention is capable of spontaneously producing a microemulsion or colloidal structures, when brought in contact, dispersed, or diluted, with an aqueous medium, e.g., water, fluids containing water, in vivo media in mammals, such as the gastric juices of the gastrointestinal tract. The colloidal structures can be solid or liquid particles including droplets, micelles and nanoparticles.

As used herein, the term “microemulsion” refers to a clear or translucent, slightly opaque, opalescent, non-opaque or substantially non-opaque colloidal dispersion that is formed spontaneously or substantially spontaneously when its components are brought into contact with an aqueous medium. A microemulsion is thermodynamically stable and contains dispersed particles, for example of a solid or liquid state (e.g., liquid lipid particles or droplets), of a mean diameter less than about 300 nm, e.g., less than about 250 nm or less than 200 nm, less than 150 nm, less than 100 nm, greater than about 2-4 nm as measured by standard light scattering techniques, e.g., using a MALVERN ZETASIZER 3000 particle characterizing machine. Solid particles in a microemulsion can be amorphous or crystalline in nature which can, for example, have particle sizes greater than 300 nm. Such microemulsions are termed overloaded microemulsion systems.

Microemulsions, e.g., are thermodynamically stable, e.g., for at least fifteen minutes, or up to four hours or even twenty-four hours or longer. As used herein the term “spontaneously dispersible” refers to a composition that is capable of producing such colloidal structures when diluted with an aqueous medium when the components of the composition of the invention are brought into contact with an aqueous medium, e.g. by simple shaking by hand for a short period of time, for example for ten seconds.”

Microemulsions can offer greater ease of preparation due to spontaneous formation, thermodynamic stability and elegant aesthetics. Microemulsions improve the delivery of the drug because they can increase drug loading, enhance penetration, reduce particle size, improve particle size uniformity, increase dissolution rate, increase bioavailability and reduce inter- and intra-individual variability in drug pharmacokinetics as compared to traditional coarse emulsions. As used herein, the term “bioavailable”, with reference to a composition, means that composition provides a maximum concentration of the drug in that composition in a use environment that is at least 1.5-fold that of a control comprising an equivalent quantity of the undispersed drug.

Without being bound to any particular theory, it is believed that by forming a microemulsion when in contact with aqueous medium, the drug delivery system minimizes the risk that the drug, especially a poorly water-soluble drug, will precipitate or crystallize out of the aqueous dispersion. Moreover, the microemulsion enhances the absorption of the drug into the mammal.

As used herein, the term “microemulsion preconcentrate” means a composition, or preconcentrate, which spontaneously forms a microemulsion, e.g., an o/w microemulsion, in an aqueous medium, in water, e.g., on dilution of 1:1 to 1:300, or from 1:1 to 1:70, or from 1:1 to 1:10 or in the gastrointestinal fluids after oral application.

The relative proportions, within the microemulsion preconcentrate, of the lipophilic component, the hydrophilic component and the surfactant lie within the “microemulsion” region on a standard three-way plot graph. Such graphs, or phase diagrams, can be generated in a conventional manner by one of ordinary skill in the art. For example, as described in Great Britain Patent No. 2,222,770, which is hereby incorporated by reference in its entirety.

A microemulsion preconcentrate, comprises a lipophilic component, a surfactant, and an optional hydrophilic component. The hydrophilic component and the surfactant together in the drug delivery system can comprise up to 95% by weight of the composition of the carrier, e.g., 80%.

As used herein, the term “solid” means a component or composition that is in a solid state at room temperature (“RT”), approximately 25-27° C., in the form of a flowable powder and having a melting point of, for example, above 40° C., e.g., up to about 65° C.

As used herein, the term “semisolid” means to a component or composition which does not flow as a powder and is not liquid at room temperature, e.g., having a melting point between room temperature and about 40° C. A semisolid can have the qualities and/or attributes of both the solid and liquid states of matter. As used herein, the term “solidify” means to make solid or semisolid.

As used herein, the term “lipophilic component” refers to a substance, material or ingredient that is more compatible with oil than with water. A material with lipophilic properties is insoluble or almost insoluble in water but is easily soluble in oil or other nonpolar solvents. The term “lipophilic component” can comprise one or more lipophilic substances. Multiple lipophilic components may constitute the lipophilic phase of the microemulsion preconcentrate and form the oil aspect, e.g., in an o/w microemulsion. At room temperature, the lipophilic component and lipophilic phase of the microemulsion preconcentrate can be solid, semisolid or liquid. For example, a solid lipophilic component can exist as a paste, granular form, powder or flake. If more than one excipient comprises the lipophilic component, the lipophilic component can be a mixture of liquids, solids, or both.

For example, the lipophilic component comprises from about 5% to about 85% by weight of the composition, e.g., from about 10% to about 85%, e.g., from about 10% to about 85%, e.g., from about 15% to about 60%, e.g., from about 20% to about 40%.

Examples of solid lipophilic components, i.e., solid or semisolid at room temperature, include, but are not limited to, the following:
1. mixtures of mono-, di- and triglycerides, such as hydrogenated coco-glycerides [melting point (m.p.) of about 33.5° C. to about 37° C.], commercially-available as WITEPSOL H15 from Sasol Germany (Witten, Germany); Examples of fatty acid triglycerides (e.g., C₁₀-C₂₂ fatty acid) triglyerides include natural and hydrogenate oils, such as vegetable oils;
2. esters, such as propylene glycol (PG) stearate, commercially-available as MONOSTEOL (m.p. of about 33° C. to about 36° C.) from Gattefossé Corp. (Paramus, N.J.); diethylene glycol palmito stearate, commercially-available as HYDRINE (m.p. of about 44.5° C. to about 48.5° C.) from Gattefossé Corp.;
3. polyglycolsylated saturated glycerides, such as hydrogenated palm/palm kernel oil PEG-6 esters (m.p. of about 30.5° to about 38° C.), commercially-available as LABRAFIL M2130 CS from Gattefossé Corp. or Gelucire 33/01;

4. fatty alcohols, such as myristyl alcohol (m.p. of about 39° C.), commercially-available as LANETTE 14 from Cognis Corp. (Cincinnati, Ohio); esters of fatty acids with fatty alcohols, e.g., cetyl palmitate (m.p. of about 50° C.); isosorbid monolaurate, e.g. commercially available under the trade name ARLAMOL ISML from Uniqema (New Castle, Del.), e.g. having a melting point of about 46° C.;
5. PEG-fatty alcohol ether, including polyoxyethylene (2) cetyl ether, e.g. commercially available as BRIJ 52 from Uniqema, having a melting point of about 33° C., or polyoxyethylene (2) stearyl ether, e.g. commercially available as BRIJ 72 from Uniqema having a melting point of about 43° C.;
6. sorbitan esters, e.g. sorbitan fatty acid esters, e.g. sorbitan monopalmitate or sorbitan monostearate, e.g. commercially available as SPAN 40 or SPAN 60 from Uniqema and having melting points of about 43 to 48° C. or about 53 to 57 and 41 to 54° C., respectively; and
7. glyceryl mono-C₆-C₁₄-fatty acid esters. These are obtained by esterifying glycerol with vegetable oil followed by molecular distillation. Monoglycerides include, but are not limited to, both symmetric (i.e. β-monoglycerides) as well as asymmetric monoglycerides (α-monoglycerides). They also include both uniform glycerides (in which the fatty acid constituent is composed primarily of a single fatty acid) as well as mixed glycerides (i.e. in which the fatty acid constituent is composed of various fatty acids). The fatty acid constituent may include both saturated and unsaturated fatty acids having a chain length of from e.g. C₈-C₁₄. Particularly suitable are glyceryl mono laurate e.g. commercially available as IMWITOR 312 from Sasol North America (Houston, Tex.), (m.p. of about 56-60° C.); glyceryl mono dicocoate, commercially available as IMWITOR 928 from Sasol (m.p. of about 33-37° C.); monoglyceryl citrate, commercially available as IMWITOR 370, (m.p. of about 59 to about 63° C.); or glyceryl mono stearate, e.g., commercially available as IMWITOR 900 from Sasol (m.p. of about 56-61° C.); or self-emulsifying glycerol mono stearate, e.g., commercially available as IMWITOR 960 from Sasol (m.p. of about 56-61° C.).

Examples of liquid lipophilic components, i.e., liquid at room temperature include, but are not limited to, the following:
1. mixtures of mono-, di- and triglycerides, such as medium chain mono- and diglycerides glyceryl caprylate/caparate, commercially-available as CAPMUL MCM from Abitec Corp. (Columbus, Ohio);
2. glyceryl mono- or di fatty acid ester, e.g. of C₆-C₁₈, e.g. C₆-C₁₆, e.g. C₈-C₁₀, e.g. C₈, fatty acids, or acetylated derivatives thereof, e.g. MYVACET 9-45 or 9-08 from Eastman Chemicals (Kingsport, Tenn.) or IMWITOR 308 or 312 from Sasol;
3. propylene glycol mono- or di-fatty acid ester, e.g. of C₆-C₂₀, e.g. C₈-C₁₂, fatty acids, e.g. LAUROGLYCOL 90, SEFSOL 218, or CAPRYOL 90 or CAPMUL PG-8 from Abitec Corp.;
4. oils, such as safflower oil, sesame oil, almond oil, peanut oil, palm oil, wheat germ oil, corn oil, castor oil, coconut oil, cotton seed oil, soybean oil, olive oil and mineral oil;
5. fatty acids or alcohols, e.g. C₆-C₂₀, saturated or mono-or di-unsaturated, e.g. oleic acid, oleyl alcohol, linoleic acid, capric acid, caprylic acid, caproic acid, tetradecanol, dodecanol, decanol;
6. medium chain fatty acid triglycerides, e.g. C₆-C₁₂, e.g. MIGLYOL 812, or long chain fatty acid triglycerides, e.g. vegetable oils;
7. transesterified ethoxylated vegetable oils, e.g. commercially available as LABRAFIL M2125 CS from Gattefossé Corp;
8. esterified compounds of fatty acid and primary alcohol, e.g. C₈-C₂₀ fatty acids and C₂-C₃ alcohols, e.g. ethyl linoleate, e.g. commercially available as NIKKOL VF-E from Nikko Chemicals (Tokyo, Japan), ethyl butyrate, ethyl caprylateoleic acid, ethyl oleate, isopropyl myristate and ethyl caprylate;
9. essential oils, or any of a class of volatile oils that give plants their characteristic odors, such as spearmint oil, clove oil, lemon oil and peppermint oil;
10. fractions or constituents of essential oils, such as menthol, carvacrol and thymol;
11. synthetic oils, such as triacetin, tributyrin;
12. triethyl citrate, acetyl triethyl citrate, tributyl citrate, acetyl tributyl citrate;
13. polyglycerol fatty acid esters, e.g. diglyceryl monooleate, e.g. DGMO-C, DGMO-90, DGDO from Nikko Chemicals; and
14. sorbitan esters, e.g. sorbitan fatty acid esters, e.g. sorbitan monolaurate, e.g. commercially available as SPAN 20 from Uniqema.

As used herein, the “hydrophilic component” comprises a hydrophilic component and/or water. A solid hydrophilic component is added in the microemulsion preconcentrate in order to render or help render the microemulsion preconcentrate a solid or semisolid at room temperature. The hydrophilic component can comprise more than one excipient. If more than one excipient comprises the hydrophilic component, the hydrophilic component can be a mixture of liquids, solids, or both.

The hydrophilic component, when present, may comprise from about 0 to about 60% by weight of the composition, e.g., from about 10% to about 50%, e.g., from about 10% to about 40%, e.g. from about 10% to about 30%.

An example of a hydrophilic component is PEG which is the polymer of ethylene oxide that conforms generally to the formula H(OCH₂CH₂)ₙOH in which n represents the average molecular weight of the polymer.

The types of PEG useful in the present invention can be categorized by its state of matter, i.e., whether the substance exists in a solid or liquid form at room temperature and pressure. As used herein, “solid PEG” refers to PEG having a molecular weight such that the substance is in a solid state at room temperature and pressure. For example, PEG having a molecular weight ranging between 1,000 and 10,000 is a solid PEG. Such PEGs include, but are not limited to PEG 1000, PEG 1550, PEG 2000, PEG 3000, PEG 3350, PEG 4000 or PEG 8000. Particularly useful solid PEGs are those having a molecular weight between 1,450 and 8,000. Especially useful as a solid PEG are PEG 1450, PEG 3350, PEG 4000, PEG 8000, derivatives thereof and mixtures thereof. PEGs of various molecular weights are commercially-available as the CARBOWAX SENTRY series from Dow Chemicals (Danbury, Conn.). Moreover, solid PEGs have a crystalline structure, or polymeric matrix, which is a particularly useful attribute in the present invention. Polyethylene oxide (“PEO”) which has an identical structure to PEG but for chain length and end groups are also suitable for use in the present invention. Various grades of PEO are commercially available as POLYOX from Dow Chemicals. PEO, for example, has a molecular weight ranging from about 100,000 to 7,000,000. The hydrophilic component in the present invention can comprise PEG, PEO, and any combinations of the foregoing.

In one exemplary embodiment of the present invention, up to eighty percent of the carrier, when liquefied, for example comprising the lipophilic component, surfactant and drug, can be incorporated into the hydrophilic component without disturbing the crystalline structure of the hydrophilic component.

The hydrophilic components of the present invention can optionally include a lower alkanol, e.g., ethanol. While the use of ethanol is not essential, it can improve drug solubility in the carrier, improve storage characteristics and/or reduce the risk of drug precipitation. The lower alkanol can comprise from 0 to about 60% by weight of the composition, e.g., from about five to about thirty percent; for example from about five to about twenty percent by weight of the composition.

In an alternative exemplary embodiment, the hydrophilic component of the carrier consists of a single hydrophilic component, e.g., a solid PEG, e.g., PEG 1450, PEG 3350, PEG 4000 and PEG 8000. In this exemplary embodiment, the hydrophilic phase of the microemulsion component consists of a single hydrophilic substance. For example, if the carrier comprised PEG 3350, the carrier would contain no other hydrophilic substances, e.g., lower alkanols (lower alkyl being C₁-C₄), such as ethanol; or water.

In yet another alternative exemplary embodiment, the hydrophilic component of the carrier consists of a mixture of solid PEGs. For example, the hydrophilic component comprises PEG 1450, PEG 3350, PEG 4000, PEG 8000, derivatives thereof and any combinations and mixtures thereof.

The carrier also comprises one or more surfactants, i.e., a mixture of surfactants; or surface active agents, which reduce interfacial tension. The surfactant is, e.g., nonionic, ionic or amphoteric. Surfactants can be complex mixtures containing side products or unreacted starting products involved in the preparation thereof, e.g., surfactants made by polyoxyethylation may contain another side product, e.g., PEG. The surfactant or surfactants can have any HLB that is useful in the pharmaceutical arts. For example, the surfactant has a hydrophilic-lipophilic balance (HLB) having a mean HLB value of 8-17, e.g., 10-17. The surfactants can also be liquid or solid in nature.

Examples of solid surfactants include, but are not limited to,
1. reaction products of a natural or hydrogenated castor oil and ethylene oxide. The natural or hydrogenated castor oil may be reacted with ethylene oxide in a molar ratio of from about 1:35 to about 1:60, with optional removal of the PEG component from the products. Various such surfactants are commercially-available, e.g., the CREMOPHOR series from BASF Corp. (Mt. Olive, N.J.), such as CREMOPHOR RH 40 which is PEG-40 hydrogenated castor oil which has a saponification value of about 50- to 60, an acid value less than about one, a water content, i.e., Fischer, less than about 2%, an n_{D}⁶⁰ of about 1.453-1.457, and an HLB of about 14-16;
2. polyoxyethylene fatty acid esters that include polyoxyethylene stearic acid esters, such as the MYRJ series from Uniqema e.g., MYRJ 53 having a m.p. of about 47° C. Particular compounds in the MYRJ series are, e.g., MYRJ 53 having a m.p. of about 47° C. and PEG-40-stearate available as MYRJ 52;
3. sorbitan derivatives that include the TWEEN series from Uniqema, e.g., TWEEN 60;
4. polyoxyethylene-polyoxypropylene co-polymers and block co-polymers or poloxamers, e.g., SYNPERONIC PE/F 87/108/127 from Uniqema;
5. polyoxyethylene alkyl ethers, e.g., such as polyoxyethylene glycol ethers of C₁₂-C₁₈ alcohols, e.g., polyoxyl 10- or 20-cetyl ether or polyoxyl 23-lauryl ether, or 20-oleyl ether, or polyoxyl 10-, 20- or 100-stearyl ether, as known and commercially-available as the BRIJ series from Uniqema. Particularly useful products from the BRIJ series are BRIJ 58; BRIJ 76; BRIJ 78; BRIJ 35, i.e., polyoxyl 23 lauryl ether; and BRIJ 98, i.e., polyoxyl 20 oleyl ether. These products have a m.p. between about 32° C. to about 43° C.;
6. water-soluble tocopheryl PEG succinic acid esters available from Eastman Chemical Co. with a m.p. of about 36° C., e.g, TPGS, e.g., vitamin E TPGS.
7. PEG sterol ethers having, e.g., from 5-35 [CH₂—CH₂—O] units, e.g., 20-30 units, e.g., SOLULAN C24 (Choleth-24 and Cetheth-24) from Chemron (Paso Robles, Calif.); similar products which may also be used are those which are known and commercially available as NIKKOL BPS-30 (polyethoxylated 30 phytosterol) and NIKKOL BPSH-25 (polyethoxylated 25 phytostanol) from Nikko Chemicals;
8. polyglycerol fatty acid esters, e.g., having a range of glycerol units from 4-10, or 4, 6 or 10 glycerol units. For example, particularly suitable are deca-/hexa-/tetra-glyceryl monostearate, e.g., DECAGLYN, HEXAGLYN and TETRAGLYN from Nikko Chemicals;
9. alkylene polyol ether or ester, e.g., lauroyl macrogol-32 glycerides and/or stearoyl macrogol-32 glycerides which are GELUCIRE 44/14 and GELUCIRE 50/13 respectively;
10. polyoxyethylene mono esters of a saturated C₁₀ to C₂₂, such as C₁₈ substituted e.g. hydroxy fatty acid; e.g. 12 hydroxy stearic acid PEG ester, e.g. of PEG about e.g. 600-900 e.g. 660 Daltons MW, e.g. SOLUTOL HS 15 from BASF (Ludwigshafen, Germany). According to a BASF technical leaflet MEF 151E (1986), SOLUTOL HS 15 comprises about 70% polyethoxylated 12-hydroxystearate by weight and about 30% by weight unesterified polyethylene glycol component. It has a hydrogenation value of 90 to 110, a saponification value of 53 to 63, an acid number of maximum 1, and a maximum water content of 0.5% by weight;
11. polyoxyethylene-polyoxypropylene-alkyl ethers, e.g. polyoxyethylene-polyoxypropylene-ethers of C₁₂ to C₁₈ alcohols, e.g. polyoxyethylen-20-polyoxypropylene-4-cetylether which is commercially available as NIKKOL PBC 34 from Nikko Chemicals;
12. polyethoxylated distearates, e.g. commercially available under the tradenames ATLAS G 1821 from Uniqema and NIKKOCDS-6000P from Nikko Chemicals; and
13. lecithins, e.g. soy bean phospholipid, e.g. commercially available as LIPOID S75 from Lipoid GmbH (Ludwigshafen, Germany) or egg phospholipid, commercially available as PHOSPHOLIPON 90 from Nattermann Phospholipid (Cologne, Germany).

Examples of liquid surfactants include, but are not limited to, sorbitan derivatives such as TWEEN 20, TWEEN 40 and TWEEN 80, SYNPERONIC L44, and polyoxyl 10-oleyl ether, all available from Uniqema.

The surfactant may comprise from about 5% to about 90% by weight of the composition of the invention, e.g. from about 15% to about 85% by weight, e.g., about 20% to about 60% by weight, e.g. from about 35% to about 55% by weight.

In certain exemplary embodiments of the present invention, the pharmaceutical composition may comprise additional excipients commonly found in pharmaceutical compositions, examples of such excipients include, but are not limited to, antioxidants, antimicrobial agents, enzyme inhibitors, stabilizers, preservatives, flavors, sweeteners and other components as described in Handbook of Pharnaceutical Excipients, Rowe et al., Eds., 4^{th} Edition, Pharmaceutical Press (2003), which is hereby incorporated by reference.

These additional excipients may comprise from about 0.05-5% by weight of the total pharmaceutical composition. Antioxidants, anti-microbial agents, enzyme inhibitors, stabilizers or preservatives typically provide up to about 0.05-1% by weight of the total pharmaceutical composition. Sweetening or flavoring agents typically provide up to about 2.5% or 5% by weight of the total pharmaceutical composition.

Examples of antioxidants include, but are not limited to, ascorbic acid and its derivatives, tocopherol and its derivatives, butyl hydroxyl anisole and butyl hydroxyl toluene. Vitamin E as α-tocopherol is particularly useful.

Each unit dosage will suitably contain from 0.1 mg and 1000 mg drug, e.g., 0.1 mg, 1 mg, 5 mg, 10 mg, 15 mg, 25 mg, 50 mg, 100 mg, 200 mg, 250 mg, 300 mg, 400 mg or 500 mg, e.g., between 5 mg and 500 mg of drug, e.g., between 10 mg and 100 mg of drug, e.g., between 20 mg and 500 mg of drug. Such unit dosage forms are suitable for administration 1-5 times daily depending upon the particular purpose of therapy, the phase of therapy and the like.

In a further aspect of the present invention, a process for preparing a spontaneously dispersible pharmaceutical composition containing a drug, especially a poorly water-soluble drug comprises the steps of bringing the drug and a carrier comprising a lipophilic component, a surfactant and a hydrophilic component into intimate admixture. For example, the drug and the carrier can be liquefied, for example, by heating to about 65° C. to about 75° C., and then solidifying by cooling to room temperature.

The carrier can be prepared separately before bringing the drug into intimate admixture with the drug. Alternatively, two or more of the components of the carrier can be mixed together with the drug.

In yet a further aspect, the invention provides a process for preparing a microemulsion containing a poorly soluble drug, which process comprises the following steps:
(a) bringing the drug and a microemulsion preconcentrate comprising a lipophilic component, a surfactant and optionally a hydrophilic component into intimate admixture to form a spontaneously dispersible pharmaceutical composition; and
(b) diluting the spontaneously dispersible pharmaceutical composition in an aqueous medium to form a microemulsion.

The compositions of the invention exhibit especially advantageous properties when administered orally, e.g., in terms of consistency and high level of bioavailability obtained in standard bioavailability trials.

Pharmacokinetic parameters, e.g., drug substance absorption and measured, e.g., as blood levels, also become surprisingly more predictable and problems in administration with erratic absorption may be eliminated or reduced. Additionally, the pharmaceutical compositions are effective with biosurfactants or tenside materials, e.g., bile salts, being present in the gastrointestinal tract. That is, the pharmaceutical compositions of the present invention are fully dispersible in aqueous systems comprising such natural tensides and thus capable of providing emulsion or microemulsion systems and/or particulate systems in situ which are stable. The function of the pharmaceutical compositions upon oral administration remain substantially independent of and/or unimpaired by the relative presence or absence of bile salts at any particular time or for any given individual. The compositions of this invention may also reduce variability in inter- and intra-patient dose response.

The utility of all the pharmaceutical compositions of the present invention may be observed in standard clinical tests in, e.g., known indications of drug dosages giving therapeutically effective blood levels of drug, e.g., using dosages in the range of 2.5-1000 mg of drug per day for a 75 kg mammal, e.g., adult and in standard animal models. The increased bioavailability of the drug provided by the compositions may be observed in standard animal tests and in clinical trials.

The following examples are illustrative, but do not serve to limit the scope of the invention described herein. The examples are meant only to suggest a method of practicing the present invention. Quantities of ingredients, represented by percentage by weight of the pharmaceutical composition, used in each example are set forth in the respective tables located after the respective descriptions.

### EXAMPLES 1 TO 3 USING CYCLOSPORINE A

The pharmaceutical compositions of Examples 1 through 3 exhibit especially advantageous properties when administered orally; for example in terms of consistency and high level of bioavailability obtained in standard bioavailability trials. These trials are performed in animals e.g. rats or dogs or healthy volunteers using HPLC or a specific or nonspecific monoclonal kit to determine the level of a poorly water soluble drug, i.e., cyclosporine A, in the blood.

For example, the compositions of Examples 1 and 2 administered p.o. to dogs can give surprisingly high Cₘₐₓ and AUC(0-24 h) values as detected by a radioimmunoassay (RIA) method using a specific monoclonal antibody and within 90 to 120% of that of NEORAL, commercially available soft gelatin capsules containing cyclosporine available from Novartis AG (Basel, Switzerland).

The compositions of all examples are prepared whereby the carrier components are mixed, liquefied, and the drug is dissolved therein whilst stirring. The mixtures are filled into hard gelatin capsules (e.g., QUALICAPS from Shionogi (Nara, Japan).

### Example 1

### Composition with a Solid Lipophilic Component

(w/w %)

Ingredients

| | |
|---|---|
| cyclosporine A | 10 |
| CREMOPHOR RH40 | 54 |
| CREMEOL FR36 (solid lipophilic component) | 27 |
| propylene glycol | 9 |

Aspect

| | |
|---|---|
| Tel quel after stirring: RT | solid |
| 1/10 in water (37° C.: water and formulation) | translucent |
| average droplet size (nm) | not measured |

### Examples 2

### Compositions with Solid Surfactants

| | | | | |
|---|---|---|---|---|
| (w/w %) | (w/w %) | (w/w %) | (w/w %) | (w/w %) |

Composition 2a Ingredients

| | | | | | |
|---|---|---|---|---|---|
| cyclosporine A | 10 | 10 | 10 | 10 | 10 |
| CREMOPHOR RH40 | 54 | 54 | 45 | 40 | 32 |
| TPGS (solid surfactant) | 13.5 | 20.25 | 18 | 24 | 32 |
| alpha tocopherol | 13.5 | 6.75 | 18 | | |
| MIGLYOL 812 | | | | 16 | 16 |
| propylene glycol | 9 | 9 | 9 | 10 | 10 |

Aspect

| | | | | | |
|---|---|---|---|---|---|
| Tel quel after stirring: RT | solid | solid | solid | solid | solid |
| 1/10 in water (37° C.: water | almost clear | almost clear | translucent | almost clear | almost clear |
| and formulation) | | | | | |
| average droplet size (nm) | 20.8 nm | 14.4. nm | 24.6 nm | n/a | n/a |

| | | | |
|---|---|---|---|
| (w/w %) | (w/w %) | (w/w %) | (w/w %) |

Composition 2b Ingredients

| | | | | |
|---|---|---|---|---|
| cyclosporine A | 5 | 5 | 5 | 5 |
| CREMOPHOR RH40 | 22.5 | 22.5 | 27 | 22.5 |
| GELUCIRE 44/14 | 50 | 50 | 50 | 50 |
| (solid surfactant) | | | | |
| corn oil glycerides | 18 | | | |
| propylene glycol | | 18 | | |
| mono-/dilaurate | | | | |
| MIGLYOL 812 | | | 9 | |
| oleyl alcohol | | | | 13.5 |
| propylene glycol | 4.5 | 4.5 | 9 | 9 |

Aspect

| | | | | |
|---|---|---|---|---|
| Tel quel after | solid | solid | solid | solid |
| stirring: RT | | | | |
| 1/10 in water | almost | almost | almost | almost |
| (37° C.: water and | clear | clear | clear | clear |
| formulation) | | | | |
| average droplet | 30.3 nm | 20.4 nm | 18.3 nm | 17.1 nm |
| size (nm) | | | | |

| | | |
|---|---|---|
| (w/w %) | (w/w %) | (w/w %) |

Composition 2c Ingredients

| | | | |
|---|---|---|---|
| Placebo | | | |
| SOLULAN C24 (solid | 60 | 70 | 50 |
| surfactant) | | | |
| MIGLYOL 812 | 10 | | |
| corn oil glycerides | | 20 | 20 |
| propylene glycol | 30 | 10 | 30 |

Aspect

| | | | |
|---|---|---|---|
| Telquel after stirring: RT | solid | solid | solid |
| 1/10 in water (37° C.: water | translucent | almost clear | almost |
| and formulation) | | | translucent |

| | | |
|---|---|---|
| (w/w %) | (w/w %) | (w/w %) |

Composition 2d Ingredients

| | | | |
|---|---|---|---|
| cyclosporine A | 10 | 10 | 10 |
| SOLULAN C24 | 54 | 63 | 54 |
| MIGLYOL 812 | 9 | | |
| corn oil glycerides | | 18 | 18 |
| propylene glycol | 27 | 9 | 18 |

Aspect

| | | | |
|---|---|---|---|
| Tel quel after stirring: RT | solid | solid | solid |
| 1/10 in water (37° C.: water | almost | slightly | slightly |
| and formulation) | translucent | opalescent | opalescent |

### Example 3

### Composition with a Solid Lipophilic Component and a Solid Surfactant

(w/w %)

Composition 3 Ingredients

| | |
|---|---|
| cyclosporine A | 6 |
| CREMOPHOR RH40 | 21 |
| SYNPERONIQUE PE L44 | 3 |
| GELUCIRE 44/14 | 40 |
| MIGLYOL 812 | 10.8 |
| LABRAFIL 2125 | 3 |
| GMOrphic 80 | 4.2 |
| Propylene glycol | 12 |

Aspect

| | |
|---|---|
| Tel quel after stirring: RT | solid |
| 1/10 in water of 37° C. | almost clear |
| average droplet size (nm) | 21.4 nm |

The mixtures are characterized by dilution 1/10 in water at 37° C. and microscopy. Light scattering, e.g. Zetasizer measurements are performed if appropriate. Stability and dissolution behavior of HGC are measured. HGC filled with compositions of the invention show excellent stability and excellent dissolution behavior, i.e. 90% of drug released after fifteen minutes.

### EXAMPLES 4 TO 5 USING COMPOUND I

Various carriers are made without the inclusion of a drug to determine whether the formulations are able to form microemulsions upon the addition of an aqueous medium, e.g., water. In each of the carriers set forth in Table 1, the microemulsion preconcentrate comprises a hydrophobic component, i.e., an oil; a surfactant; and a hydrophilic component, e.g., PEG 3350. The ratio of the PEG 3350: hydrophobic component:surfactant is 4:3:3. Thus, each carrier comprises 40% PEG 3350; 30% hydrophobic component, i.e., an oil; and 30% surfactant by weight of the microemulsion preconcentrate, or carrier.

To make each carrier, the components are added to a water-jacketed beaker and heated to about 65° C. to about 75° C. Agitation of the ingredients is accomplished by the use of a magnetic stir bar. Once the components are homogeneously melted and mixed, the composition is filled into hard gelatin capsules, e.g., size 0, using a positive displacement pipette. During the filling of the capsules, the melted carrier composition is maintained at 65° C. Thus, the capsules are manufactured by a “hot melt fill” method. Once filled, the carrier composition is allowed to solidify for about 20-25 minutes.

The comments in Table 1 are subjective observations of the solid or semisolid forms of various microemulsion preconcentrates at room temperature. Only surfactants and oil are shown in Table 1, and a hydrophilic component PEG3350 (40%) is also present. All of the microemulsion preconcentrates in Table 1 are suitable for pharmaceutical administration.

TABLE 1

| | Oil | | | | |
|---|---|---|---|---|---|
| | | | | Refined | Essential |
| | | CAPMUL MCM | | sesame oil | spearmint oil |
| | CAPMUL PG-8 | (mono- and | CAPTEX 335 | (refined | (unrefined |
| Surfactant | (PG ester) | diglycerides) | (triglycerides) | triglycerides) | triglycerides) |
| TWEEN 60 | hard solid | no test | soft semisolid | soft semisolid | no test |
| TWEEN 80 | hard solid | hard solid | soft semisolid | soft semisolid | hard solid |
| CREMOPHOR | hard solid | hard solid | soft semisolid | soft semisolid | hard solid |
| EL | | | | | |

In the carrier comprising PEG3350/CAPMUL PG-8/CREMOPHOR EL, the melting point of the composition is about 50.7° C. as measured by differential scanning calorimetry. The particle size upon dilution with water (in a ratio of 1:250) is about 70-100 nm. The particle size is measured using a Beckman Coulter N4 Plus Submicron Particle Size Analyzer available from Beckman Coulter (Hialeah, Fla.).

In the following examples, a drug, a compound of formula (I) (hereinafter “Compound I”), is used to make a microemulsion preconcentrate of the present invention (hereinafter, “Sample 4A”) and a solid dispersion composition as a comparative example (hereinafter, “Sample 4B”).

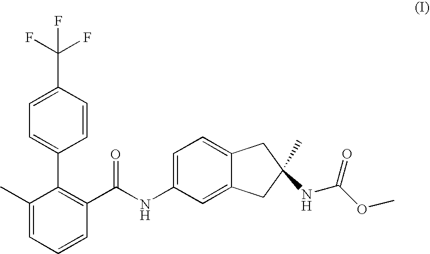

Compound I has a solubility of 0.17 μg/mL and a Log P in octanol of 4.66. The melting range of Compound I is from 120.5-151° C. In both Samples 4A and 4B, the drug loading is 4% or 20 mg/capsule. The compositions of both Sample 4A and 4B are set forth in Table 2.

TABLE 2

| | Sample 4A | |
|---|---|---|
| | (solid microemulsion | Sample 4B |
| | preconcentrate) | (solid dispersion) |
| | (w/w %) | (w/w %) |
| Compound I | 4% | 4% |
| CAPMUL PG8 | 28.8% | |
| CREMOPHOR ELP | 28.8% | |
| PEG 3350 | 38.4% | 72% |
| TWEEN 80 | | 24% |

Sample 4A is manufactured in the same method as disclosed previously for the compositions listed in Table 1. However, once the carrier components are melted, the drug is dissolved into the carrier under agitation. The process of Sample 4B is similar to that of Sample 4A, however, no hydrophobic component is added, e.g., there is no oil—CAPMUL PG8.

Sample 4A, upon dilution with water, forms a microemulsion resulting in mean particle sizes of about 70-100 nm as measured by a Beckman Coulter N4 Plus Particle Sizer. Sample 4B has mean particle sizes greater than 250 nm. Another major difference between Sample 4A and Sample 4B is that the particles formed after mixing Sample 4A with water are those of the oil phase of a microemulsion; the drug remains dissolved in the oil phase and there is no phase separation of the drug either as oily liquids or crystals. In contrast, the particles formed after mixing of the solid dispersion (Sample 4B) with water are those of the drug.

The dissolution of Sample 4A is then compared to Sample 4B using a Distek Dissolution System Model 2100A (North Brunswick, N.J.). Capsules of each sample are placed in 250 mL water at 37° C. Paddles are rotated at 75 rpm with samples being taken at 10, 20, 30, 60 and 120 minutes. The dissolution profile of Sample 4A is superior to that of Sample 4B. At approximately the 20-minute interval, more than 80% of Compound I is released in Sample 4A. In contrast, at the corresponding interval, less than 40% of Compound I is released in Sample 4B. Furthermore, during the entire dissolution test, the percentage of Compound I in Sample 4B released remains below 40% whereas for Sample 4B, the percentage of Compound I release exceeds 80%. Thus, the formulation of Sample 4A, a microemulsion, has better dissolution then Sample4B.

The differences between Sample 4A and Sample 4B become more pronounced when the particle sizes of the dissolution fluids are measured as a function of time. The mean particle size of the microemulsion formed from Sample 4A remains unchanged for up to 120 minutes while the particle size of the phase separated drug from Sample 4B increases with time. The phase-separated drug from Sample 4B exists as crystals having a size greater than 1 micron.

The differences between a solid/semisolid microemulsion preconcentrate becomes more pronounced when the drug loads in Sample 4A and Sample 4B are increased to 10% (w/w) to prepare Sample 5C and Sample 5D shown respectively in Table 3.

TABLE 3

| | Sample 5C | |
|---|---|---|
| | (solid microemulsion | Sample 5D |
| | preconcentrate) | (solid dispersion) |
| | (w/w %) | (w/w %) |
| Compound I | 10% | 10% |
| CAPMUL PG8 | 27% | |
| CREMOPHOR ELP | 27% | |
| PEG 3350 | 36% | 67.5% |
| TWEEN 80 | | 22.5% |

The particle size of the microemulsion formed by Sample 5C is still low and around 200 nm throughout the 120-minute period. In contrast, the particle size of the drug separated from the solid dispersion is much greater than one micron. Large crystals of the drug are evident from Sample 5D when the dissolution fluids are kept standing for over two hours, while no such crystals are evident from the microemulsion formed from Sample 5C. Thus, the solid microemulsion preconcentrate demonstrates a superior performance as a drug delivery system with respect to particle size distribution and particle size stability as compared to a solid dispersion even though both systems contain PEG (a hydrophilic component) and a surfactant. The presence of the liquid lipophilic component to form a microemulsion results in a major and surprising difference.

The microemulsion preconcentrate also provides superior physical stability as a drug product over the solid dispersion. There is no crystallization of the drug in the solid microemulsion preconcentrate with the drug load up to 8% immediately after preparation and upon storage under various accelerated stability storage conditions for a period of at least one month. In comparison, crystals are observed when the drug load in the solid dispersion is increased above 4%.

It is understood that while the present invention has been described in conjunction with the detailed description thereof that the foregoing description is intended to illustrate and not limit the scope of the invention, which is defined by the scope of the following claims. Other aspects, advantages and modifications are within the scope of the claims.

1. A pharmaceutical composition comprising a poorly water soluble drug; and a carrier comprising (1) a lipophilic component, (2) a surfactant, and optionally (3) a hydrophilic component, wherein at least one of the components (1) to (3) is solid at room temperature, wherein said pharmaceutical composition is spontaneously dispersible.

2. The pharmaceutical composition of claim 1, wherein said hydrophilic component is a solid polyethylene glycol (PEG).

3. The pharmaceutical composition of claim 1, wherein said hydrophilic component comprises at least one chosen from PEG 1000, PEG 1550, PEG 2000, PEG 3000, PEG 4000, PEG 8000, derivatives and mixtures thereof.

4. The pharmaceutical composition of claim 1, wherein said lipophilic component is a liquid lipophilic component.

5. The pharmaceutical composition of claim 4, wherein said liquid lipophilic component is an essential oil.

6. The pharmaceutical composition of claim 1, wherein said lipophilic component is a liquid and said surfactant is solid.

7. The pharmaceutical composition of claim 1 in the form of a microemulsion upon dilution with an aqueous medium.

8. The pharmaceutical composition of claim 1, wherein said microemulsion comprises particles having a mean particle size less than 300 nm.

9. A method of treatment of a subject suffering from a disorder treatable with a poorly soluble drug comprising administering a therapeutically effective amount of a pharmaceutical composition as claimed in any preceding claim to a subject in need of such treatment.

10. A process for preparing a microemulsion containing a poorly soluble drug comprising the steps of
(a) bringing the poorly soluble drug and a liquefied carrier comprising (1) a lipophilic component, (2) a surfactant, and optionally (3) a hydrophilic component, wherein at least one of the components (1) to (3) is solid at room temperature into intimate admixture;
(b) cooling said poorly soluble drug and said carrier to form a spontaneously dispersible pharmaceutical composition; and
(c) diluting the spontaneously dispersible pharmaceutical composition in an aqueous medium to form the microemulsion.

11. A drug delivery system for a poorly soluble drug comprising a surfactant, a lipophilic component, and a hydrophilic component, wherein said hydrophilic component consists essentially of a solid PEG.

12. Use of a lipophilic component, a surfactant, or a hydrophilic component, wherein at least one component is solid at room temperature to solidify a spontaneously dispersible pharmaceutical composition.

13. The pharmaceutical composition of claim 1 wherein said surfactant is TPGS.
